(19) United States
(12) Patent Application Publication
(10) Pub. No.: US 2018/0279899 A1
(43) Pub. Date: Oct. 4, 2018

(54) SYSTEM, APPARATUS, AND METHODS FOR ACHIEVING FLOW STATE USING BIOFEEDBACK

(71) Applicant: International Business Machines Corporation, Armonk, NY (US)

(72) Inventors: Asaf Adi, Kiryat Ata (IL); Nir Mashkif, Ein Ayala (IL); Daniel Rose, Tel Aviv (IL); Alexander Zadorojniy, Haifa (IL); Sergey Zeltyn, Haifa (IL)

(21) Appl. No.: 15/477,122

(22) Filed: Apr. 3, 2017

(51) Int. Cl. A61B 5/0482; A61B 5/11; G06F 19/00; A61B 5/16; A61B 5/00; A61B 5/024; A61B 5/0205

(52) U.S. Cl. CPC A61B 5/0482; A61B 5/1118; G06F 19/3431; A61B 5/165; A61B 5/7435; G06F 19/34; A61B 5/0205; A61B 5/024; A61B 5/6801; G06F 19/3481; A61B 5/02438

(57) ABSTRACT

A system having a wearable devices that, together with a cognitive model, are able to analyze a person to determine if they are in the flow and/or guide the person to get into the flow are disclosed. The system and processes help persons to find their unique formula to achieve flow. By using a cognitive AI engine, the system can describe a space of mental states and the actions that cause transitions between them for each individual.

### FIELD OF TECHNOLOGY

The present invention relates to the technical fields of wearable and cognitive systems. In particular, the present invention relates to biofeedback signals and their interpretation.

### BACKGROUND OF THE INVENTION

For many persons, there are times when they are not on top of their game even though they feel they should be. At other times, despite not feeling up to a task, a person will perform his or her best. For example, in sports, an athlete may get sick and be unable to train properly. Despite the lack of training, the athlete may be able to perform their best. In contrast, an athlete may have what is conventionally considered to be good preparation, but perform poorly in competition.

Often, an athlete's coach will describe the results as an effect due to expectations. Getting into the “flow” is very challenging, even for the most elite athletes. The flow is a certain mental state that each person achieves with a different recipe to find the just right balance of confidence, tension, stress, and task focus.

The current state-of-the-art to achieve the flow is based on the work of medical doctors together with experts in sports human psychology. In practice, it comes down to a personalized set of rules and guidelines. Most commonly, athletes build formulas from personal habits, repetition of key phrases, or other specific behaviors, that take them into the flow. Often, these formulas are focused on “feelings.” When an athlete succeeds, a coach will ask him or her to remember the feeling so that he or she can recall it the next time. This approach can work but is very difficult to achieve consistent results. Even if an athlete can describe a certain “feeling” (which is itself not a trivial task) and find ways to trigger it, it is nearly impossible for the athlete to actually “know” that he in fact has achieved the state of flow until after the activity is already completed.

On the other hand, objective techniques to improve performance rely on biofeedback systems that monitor heart rate and skin conductivity. This data can describe physically what the body looks like when it's in the flow, and can help people get closer to it. For example, if an athlete performs best when his heart rate is 130 beats/min, and his heartbeat is now 85 beats/min, the athlete should increase his heart rate. However, this by itself will not usually place the athlete into the flow. The flow is a mental state that is more complicated to achieve than simply undertaking a certain physical activity. An athlete's thoughts, fears, and desires all impact his or her ability to achieve the flow at a specific moment.

While there has been some research at creating models for “mental state” such as moods, conventional techniques have not successfully created a model that can describe “flow” or that find policies for transitioning between mental states to achieve the flow. Accordingly, a need arises for techniques that can successfully create a model that can describe “flow” and find policies for transitioning between mental states to achieve the flow.

### SUMMARY OF INVENTION

In embodiments, the present invention relates to wearable devices that, together with a cognitive model, are able to analyze a person to determine if they are in the flow and/or guide the person to get into the flow.

In embodiments, the processes disclosed help persons to find their unique formula to achieve flow. By using a cognitive AI engine, the system described herein can describe a space of mental states and the actions that cause transitions between the states for each individual.

In alternative embodiments, the present invention is a vertical solution targeted at the weekend warriors, i.e., semi-professional and professional athletes who want to improve their performance.

In embodiments, a system for guiding a person to achieve the flow, the system comprises a first sensor configured to receive an input signal from a test subject during performance of an activity, and a server comprising a sensor interface coupled to the first sensor, a user interface, a microprocessor, and a memory that stores a set of actions, where the microprocessor defines a set of unobservable states, where in at least one state in the set of states represents a flow state, defines a set of observations, defines a metric for comparison of states and actions, defines a cost for each pair of states and actions, constructs a graph based on the unobservable states, a set of transitions between the states, the actions, and the costs, constructs transitions between unobservable states and observations, initializes the graph transitions uniformly or based on a set of domain knowledge, computes a policy utilizing the graph, where the policy specifies an action from the set of actions to be taken, and transmits the policy to the user interface.

In an optional embodiment, the first sensor is selected from the group consisting of a heart rate sensor and a galvanic skin response sensor. In an alternative embodiment, the first sensor is selected from the group consisting of an accelerometer and a gyroscope. In a preferred embodiment, the system further comprises a second sensor. In another preferred embodiment, the graph comprises a Partially Observable Markov Decision Process model (POMDP).

In an optional embodiment, the system further comprises displaying a visual cue to a display based on the solution to the problem. In another optional embodiment, generating at least one recommended action per each state from the set of actions comprises transmitting a haptic cue. In an alternative embodiment, the microprocessor is further configured to provide full state information and traversed path of states, including state transitions. In a preferred embodiment, solving the problem comprises executing a value iterations algorithm or any other algorithm for solving of POMDP. In an optional embodiment, the microprocessor initializes a conditional probability of observations using either domain knowledge or uniformly.

Numerous other embodiments are described throughout herein. All of these embodiments are intended to be within the scope of the invention herein disclosed. Although various embodiments are described herein, it is to be understood that not necessarily all objects, advantages, features or concepts need to be achieved in accordance with any particular embodiment. Thus, for example, those skilled in the art will recognize that the invention may be embodied or carried out in a manner that achieves or optimizes one advantage or group of advantages as taught or suggested herein without necessarily achieving other objects or advantages as may be taught or suggested herein.

The methods and systems disclosed herein may be implemented in any means for achieving various aspects, and may be executed in a form of a machine-readable medium embodying a set of instructions that, when executed by a machine, cause the machine to perform any of the operations disclosed herein. These and other features, aspects, and advantages of the present invention will become readily apparent to those skilled in the art and understood with reference to the following description, appended claims, and accompanying figures, the invention not being limited to any particular disclosed embodiment(s).

### BRIEF DESCRIPTION OF THE DRAWINGS

S₀ that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and the invention may admit to other equally effective embodiments.

FIG. 1 illustrates a system diagram, according to an embodiment.

FIG. 2 illustrates a graph model, according to an embodiment.

FIG. 3 illustrates mental states described in terms of challenge level and skill level, according to an embodiment.

FIG. 4 illustrates a flow chart illustrating a process executed by the system, according to an embodiment.

FIG. 5 illustrates a flow chart illustrating a process executed by the system, according to an alternative embodiment.

Other features of the present embodiments will be apparent from the Detailed Description that follows.

### DETAILED DESCRIPTION OF THE EMBODIMENTS

In the following detailed description of the preferred embodiments, reference is made to the accompanying drawings, which form a part hereof, and within which are shown by way of illustration specific embodiments by which the invention may be practiced. It is to be understood that other embodiments may be utilized and structural changes may be made without departing from the scope of the invention. Electrical, mechanical, logical and structural changes may be made to the embodiments without departing from the spirit and scope of the present teachings. The following detailed description is therefore not to be taken in a limiting sense, and the scope of the present disclosure is defined by the appended claims and their equivalents.

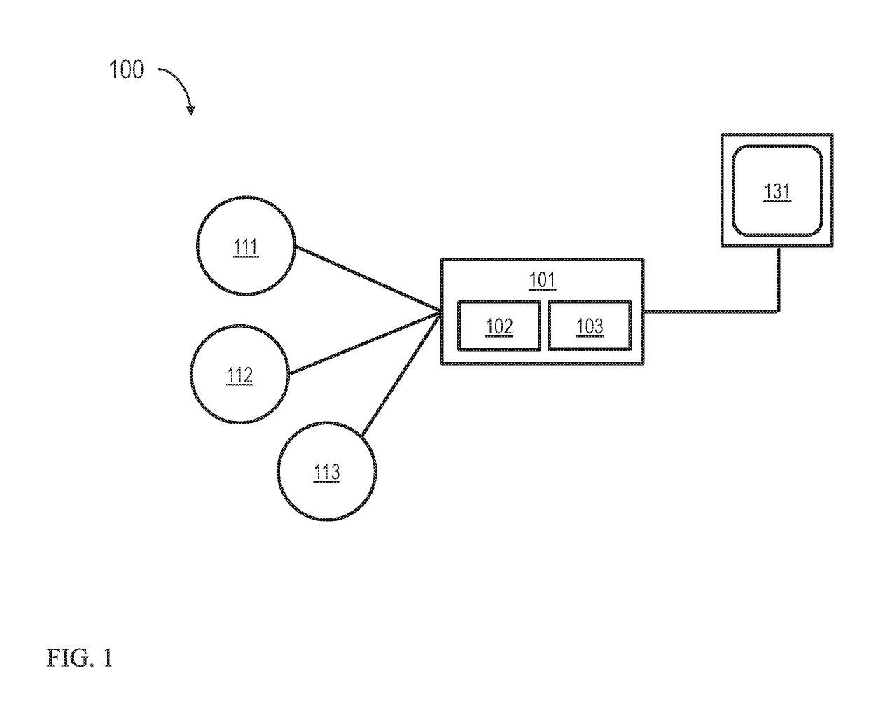

FIG. 1 illustrates a system diagram, according to an embodiment. The system 100 is comprised of a set of sensors 111, 112, 113, a control device 101, and a user interface such as dashboard, monitor, point of access, etc. 131. The control device 101 can be implemented using computer hardware and can include a processor 102 and a memory 103 that stores the software instructions, sensor data, and other data. The control device 101 can also include network interfaces, such as Wi-Fi and cellular interfaces (not shown). The sensors 111, 112, 113 can be any type of sensor including, but not limited to, heart rate sensors, galvanic skin response sensors, accelerometers, gyroscopes, magnetic compasses, microphones, pressure sensors, electroencephalograph (EEG) sensors, electrocardiograph (EKG or ECG) sensors, and temperature sensors. The sensor 111, 112, 113 output can be discretized before or at the control device 101. The control device 101 can contain a processor and memory. The user interface 131 can be any user interface device, such as a display or a touch or haptic feedback device.

The system, in embodiments, may use a model of mental states. The model can relate to those used for “focus.” The models for focus are broadly applicable. For example, the models can be used to develop a solution to address the management of medication in ADHD. The market for ADHD treatments is almost $10 billion, and many doctors say that their greatest challenge with ADHD is in setting the correct levels of medication. In fact, flow can be a better metric to observe than focus because athletes may demonstrate clearer signs of being in focus than the general population. Similarly, the use of “focus” in a model is useful in professional settings, and can be used in conjunction with IoT backends.

For describing mental states, a goal of the system, in embodiments, is that the cognitive engine operates using only relatively “simple” sensor data from wearable devices, such as a wristband, that collect inputs such as heart rate (HR), heart rate variability (HRV), and galvanic skin response (GSR). However, to build an accurate initial model, the system may use other advanced sensors such as EEG. The input to the system can also include a questionnaire as an input.

A complexity of this problem is the inherent differences in people. For example, the model of mental states almost certainly depends on the type of a subject (e.g., athlete, student, etc.) and on the personality (e.g., extrovert or introvert) of a subject. To address this, the personality insights analytics may also be used to build the initial models.

To identify which mental state represents the “flow,” empirical experiments with study subjects may be conducted. This may be measured conventionally using race results or points scored, identifying the flow according to when the athlete meets or exceed his or her own personal best results.

The actions in the cognitive model may correspond to the formulas that subjects (e.g., athletes) may have already practiced to bring them into the flow: personal habits, repetition of key phrases, or other specific behaviors. The model is then trained to understand how these actions cause transitions between the mental states. In embodiments, the engine supports customization of actions for each athlete, as each of their actions will be different.

A challenge in developing the cognitive AI engine is finding the optimal policy (e.g., the set of actions for any state) that cause the transitions required between mental states to reach the flow. This is an optimization problem that can be formulated as the Partially Observable Markov Decision Process (POMDP) framework.

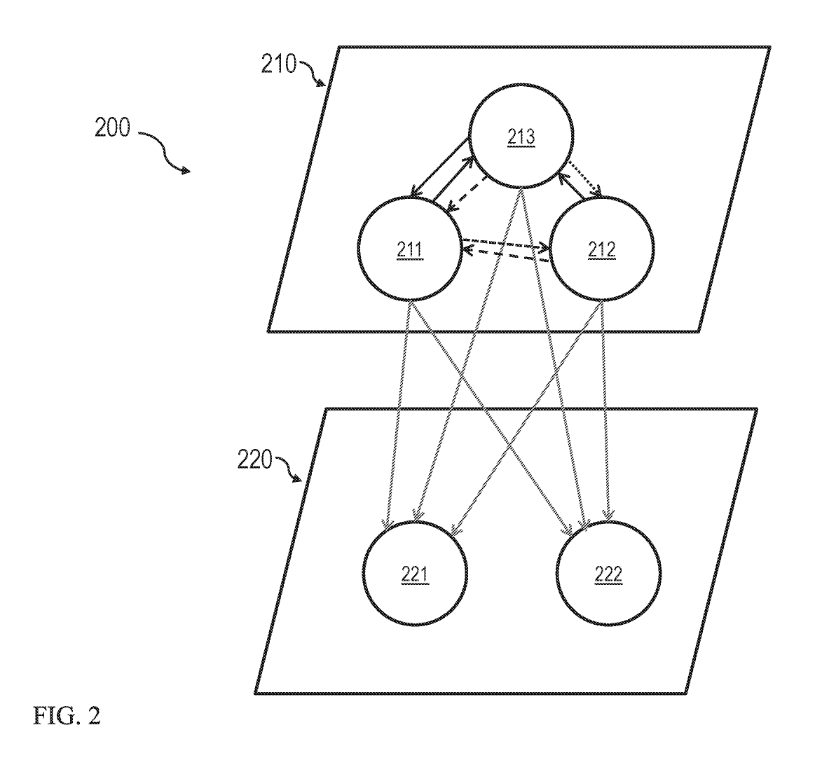

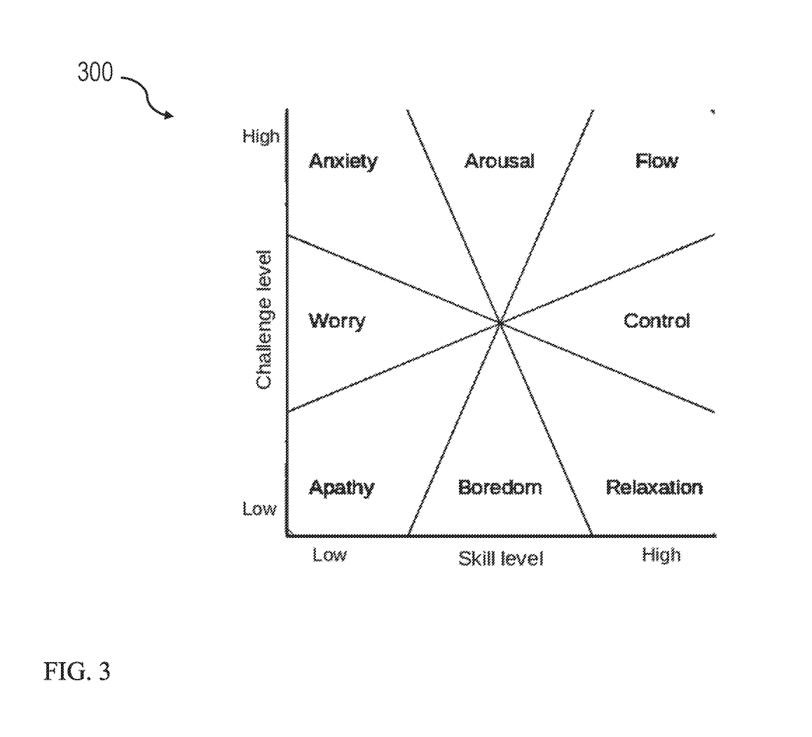

FIG. 2 illustrates a graph model 200, according to an embodiment. The graph model 200 contains a transitions graph 210 that is representative of the mental layer. A sensor layer 220 is also provided. The mental layer contains the various states 211, 212, 213. For example, the states may be the up state 211, the down state 212, and the flow state 213. Examples of other possible states may include anxiety, arousal, worry, control, apathy, boredom, and relaxation. An example of a set of mental states 300 described in terms of challenge level and skill level is shown in FIG. 3. The flow state represents the mental state of operation in which a person performing an activity is fully immersed in a feeling of energized focus, full involvement, and enjoyment in the process of the activity. For example, the flow state is further described at https://en.wikipedia.org/wiki/Flow_%28psychology%29, which is incorporated by reference in its entirety.

Returning to FIG. 2, the states have transitions between them, and the transitions can be probabilistic. Again, these states can be any of those defined shown in FIG. 3 or other state graphs can be used. There can be different types of transitions between the states, as the varying dash types of the lines represent. There can be multiple different transitions from a state to another state. Each transition can have an associated probability and represent different actions taken. If state S₁ is the state that is being transitioned to, state S₀ is the state being transitioned from, u₀ is the action taken at state S₀, then P(S₁, S₀, u₀) is the probability to move to state S₁ from S₀ when u₀ is taken at state S₀. Each transition is action dependent, and there are costs associated with each transition. The states are unobservable. The sensor layer 220 contains the sensor data 221, 222. For example, the sensor layer 220 may contain the galvanic skin response samples 221 and the heart rate samples 222. The observations have probability distributions O(o₁|S₁, u₀). Other data may be input from a user interface, such as questionnaire data. The system can use the graph model to recommend an action at each state to achieve the flow or get closer to the flow.

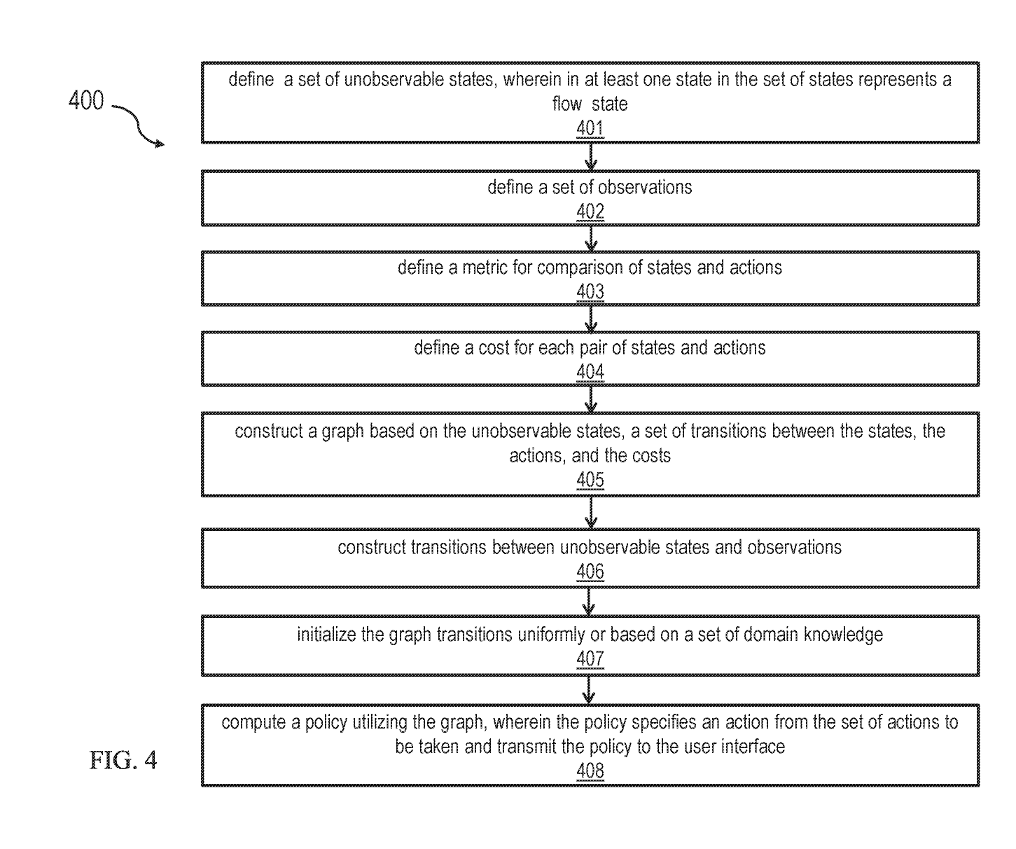

The process executed by the system, in an embodiment, is summarized in the flow chart 400 shown in FIG. 4. In step 401, a set of unobservable states are setup in the system. At least one state in the set of states represents a flow state. The set of unobservable states, such as those detailed above and in FIG. 3, can be defined by a subject matter expert for inclusion in the system. In step 402, the system or subject matter expert defines a set of observations. In step 403, the system or subject matter expert defines a metric for comparison of states and actions. In step 404, the system or subject matter expert defines a cost for each pair of states and actions. In step 405, the system or subject matter expert constructs a graph based on the unobservable states, a set of transitions between the states, the actions, and the costs. In step 406, the system or subject matter expert constructs transitions between unobservable states and observations. In step 407, the system initializes the graph transitions uniformly or based on a set of domain knowledge. In step 408, the system computes a policy utilizing the graph, wherein the policy specifies an action from the set of actions to be taken and transmits the policy to a user interface.

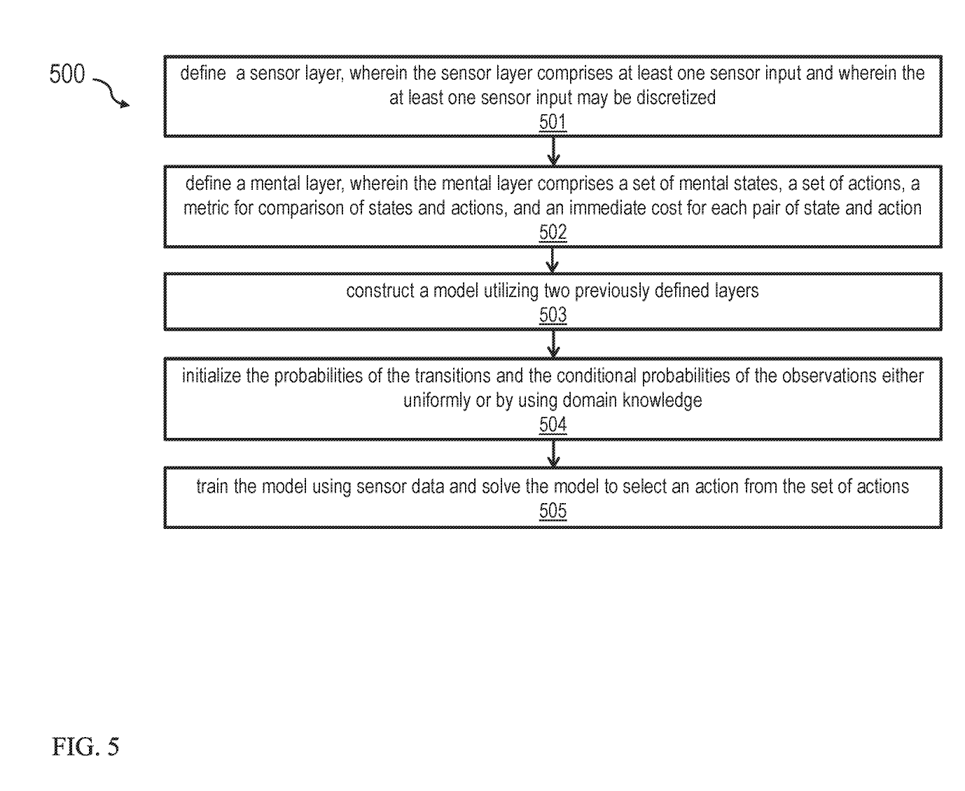

FIG. 5 illustrates another flowchart 500 illustrating a process executed by the system, according to an embodiment. In step 501, the system defines a sensor layer, wherein the sensor layer comprises at least one sensor input and wherein the at least one sensor input may be discretized. In step 502, the system defines a mental layer, wherein the mental layer comprises a set of mental states, a set of actions, a metric for comparison of states and actions, and an immediate cost for each pair of state and action. In step 503, the system constructs a model utilizing two previously defined layers. In step 504, the system initializes the probabilities of the transitions and the conditional probabilities of the observations either uniformly or by using domain knowledge. In step 505, the system trains the model using sensor data; and solves the model to select an action (either in optimal or approximate fashion) from the set of actions.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While the foregoing written description of the invention enables one of ordinary skill to make and use what is considered presently to be the best mode thereof, those of ordinary skill will understand and appreciate the existence of alternatives, adaptations, variations, combinations, and equivalents of the specific embodiment, method, and examples herein. Those skilled in the art will appreciate that the within disclosures are exemplary only and that various modifications may be made within the scope of the present invention. In addition, while a particular feature of the teachings may have been disclosed with respect to only one of several implementations, such feature may be combined with one or more other features of the other implementations as may be desired and advantageous for any given or particular function. Furthermore, to the extent that the terms “including”, “includes”, “having”, “has”, “with”, or variants thereof are used in either the detailed description and the claims, such terms are intended to be inclusive in a manner similar to the term “comprising.”

Other embodiments of the teachings will be apparent to those skilled in the art from consideration of the specification and practice of the teachings disclosed herein. The invention should therefore not be limited by the described embodiment, method, and examples, but by all embodiments and methods within the scope and spirit of the invention. Accordingly, the present invention is not limited to the specific embodiments as illustrated herein, but is only limited by the following claims.

What is claimed is:

1. A system for guiding a person to achieve the flow, the system comprising:
a first sensor configured to receive an input signal from a test subject during performance of an activity; and
a server comprising a sensor interface coupled to the first sensor, a user interface, a microprocessor, and a memory that stores a set of actions, wherein the microprocessor:
defines a set of unobservable states, wherein in at least one state in the set of states represents a flow state;
defines a set of observations;
defines a metric for comparison of states and actions;
defines a cost for each pair of states and actions;
constructs a graph based on the unobservable states, a set of transitions between the states, the actions, and the costs;
constructs transitions between unobservable states and observations;
initializes the graph transitions uniformly or based on a set of domain knowledge;
computes a policy utilizing the graph, wherein the policy specifies an action from the set of actions to be taken; and
transmits the policy to the user interface.

2. The system of claim 1, wherein the first sensor is selected from the group consisting of a heart rate sensor and a galvanic skin response sensor.

3. The system of claim 1, wherein the first sensor is selected from the group consisting of an accelerometer and a gyroscope.

4. The system of claim 1, further comprising a second sensor.

5. The system of claim 1, wherein the graph comprises a partially observable Markov decision process model.

6. The system of claim 1, further comprising:
displaying a visual cue to a display based on the solution to the problem.

7. The system of claim 1, wherein the action to be taken at each state in the unobservable states generating at least one recommended action per each state from the set of actions comprises transmitting a haptic cue.

8. The system of claim 1, wherein the microprocessor is further configured to provide full state information and traversed path of states including state transitions.

9. The system of claim 1, wherein solving the problem comprises executing a value iterations algorithm or any other algorithm for solving of POMDP.

10. The system of claim 1, wherein the microprocessor initializes a conditional probability of observations using either domain knowledge or uniformly.

11. An apparatus for guiding a test subject to achieve the flow state comprising:
at least one sensor selected from the group consisting of a heart rate sensor, a galvanic skin response sensor, an accelerometer, and a gyroscope;
a user interface;
a controller coupled to the user interface and the sensor and comprising a microprocessor and a memory that stores a set of actions, a graph of unobservable mental states, and a set of transitions between the states and observations, and wherein the microprocessor
maps the sensor to the observations,
defines a cost for each transition,
initializes the graph based on either domain knowledge or uniformly,
solves the graph to generate at least one action output from the set of actions, and
transmits the at least one action output to the user interface.

12. The apparatus of claim 11, wherein the graph comprises a partially observable Markov decision process model.

13. The apparatus of claim 12, wherein solving the partially observable Markov decision process model comprises executing a value iterations algorithm.

14. The apparatus of claim 11, wherein transmitting the at least one action output to the user interface comprises displaying a visual cue to a display.

15. The apparatus of claim 11, wherein transmitting the at least one action output to the user interface comprises displaying a visual cue to a display based on the solution to the problem.

16. The apparatus of claim 11, wherein transmitting the at least one action output to the user interface comprises transmitting a haptic cue.

17. A method for guiding a test subject to achieve the flow state comprising:
defining a sensor layer, wherein the sensor layer comprises at least one sensor input and wherein the at least one sensor input may be discretized;
defining a mental layer, wherein the mental layer comprises a set of mental states, a set of actions, a metric for comparison of states and actions, and an immediate cost for each pair of state and action;
constructing a model utilizing two previously defined layers;
initializing the probabilities of the transitions and the conditional probabilities of the observations either uniformly or by using domain knowledge;
training the model using sensor data; and
solving the model to select an action, in optimal or approximate fashion, from the set of actions.

18. The method of claim 17, wherein the sensor data comprises heart rate sensor data.

19. The method of claim 17, wherein the sensor data comprises galvanic skin response sensor data.

20. The method of claim 17, wherein the sensor data comprises accelerometer data.
